(19) United States
(12) Patent Application Publication
(10) Pub. No.: US 2023/0162824 A1
(43) Pub. Date: May 25, 2023

(54) L AND D INTEGRATED MEDICAL MODEL

(71) Applicant: Whole Child Pediatrics Natalie Drummond LTD, Yorkville, IL (US)

(72) Inventor: Marc Drummond, Naperville, IL (US)

(73) Assignee: Whole Child Pediatrics Natalie Drummond LTD, Yorkville, IL (US)

(21) Appl. No.: 17/994,110

(22) Filed: Nov. 25, 2022

Related U.S. Application Data
(60) Provisional application No. 63/282,797, filed on Nov. 24, 2021.

(51) Int. Cl. G16H 10/60; G16H 20/10

(52) U.S. Cl. CPC G16H 10/60; G16H 20/10

(57) ABSTRACT

A medical management system including an information gathering unit that gathers information on a patient, an information analysis unit that analyzes the information of each patient, a coordination unit that disseminates the patient information to different professionals, a scheduling unit that coordinates the patient visit schedules where the patient information includes medical, psychological and pharmaceutical assessments.

### CROSS REFERENCE TO RELATED APPLICATIONS

This application claims the benefit of and the priority from U.S. Provisional Patent Application No. 63/282,797, filed Nov. 24, 2021, titled L & D Integrated Medial Model, which is incorporated herein by reference.

### BACKGROUND OF THE INVENTION

The medical industry currently operates in silos where psychology, general medicine and pharmacology do not communicate. This methodology of operating independently reduces patient care and results in high medical costs. In addition, patients are often prescribed medications that have an adverse effect of vitamins and minerals in the patients body. The isolation of different medical professions results in no central knowledgebase for patent records and medical information.

A need exists for a system that treats all aspects of patient's health holistically.

### SUMMARY OF THE INVENTION

Systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

One embodiment of the present disclosure includes a medical management system including an information gathering unit that gathers information on a patient, an information analysis unit that analyzes the information of each patient, a coordination unit that disseminates the patient information to different professionals, a scheduling unit that coordinates the patient visit schedules where the patient information includes medical, psychological and pharmaceutical assessments.

In another embodiment, the environmental information related to the patient is gathered.

In another embodiment, the environmental information includes information on the environment where the patient works and lives.

In another embodiment, the environmental information includes demographic information related to the area where the patient works and lives.

In another embodiment, the patient information includes a listing of all pharmaceuticals and supplements taken by the patient.

In another embodiment, the patient information includes psychological assessments made by a trained psychologist.

In another embodiment, the patient information is regularly updated by medical professionals.

In another embodiment, the patient information includes information on the nutritional status of the patient.

In another embodiment, the patient information is normalized to similar patients.

In another embodiment, a pharmacist reviews the patient medication and makes adjustments based on information from a psychologist and physician.

Another embodiment of the present disclosure includes a method of managing a patient's overall health including the steps of gathering information on a patent an information gathering unit, analyzing the patient information via an information analysis unit, disseminating patient information to different professionals via a coordination unit, coordinating patient visits via a scheduling unit where the patient information includes medical, psychological and pharmaceutical assessments.

In another embodiment, the environmental information related to the patient is gathered.

In another embodiment, the environmental information includes information on the environment where the patient works and lives.

In another embodiment, the environmental information includes demographic information related to the area where the patient works and lives.

In another embodiment, the patient information includes a listing of all pharmaceuticals and supplements taken by the patient.

In another embodiment, the patient information includes psychological assessments made by a trained psychologist.

In another embodiment, the patient information is regularly updated by medical professionals.

In another embodiment, the patient information includes information on the nutritional status of the patient.

In another embodiment, the patient information is normalized to similar patients.

In another embodiment, a pharmacist reviews the patient medication and makes adjustments based on information from a psychologist and physician.

### BRIEF DESCRIPTION OF THE DRAWINGS

The accompanying drawings, which are incorporated in and constitute a part of this specification, illustrate an implementation of the present invention and, together with the description, serve to explain the advantages and principles of the invention. In the drawings:

FIG. 1 depicts one embodiment of an integrated medical management system consistent with the present invention;

FIG. 2 depicts one embodiment of a medical management unit 102;

FIG. 3 depicts one embodiment of a communication device consistent with the present invention;

FIG. 4 depicts a schematic representation of the components of the integrated medical management system;

FIG. 5 depicts a schematic representation of an intake process for a patient;

FIG. 6 depicts a schematic representation of a process used to enhance a patent profile;

FIG. 7 depicts a schematic representation of a process used generate a health plan;

FIG. 8 depicts a schematic representation of a process used to gather psychological information on a patient;

FIG. 9 depicts a schematic representation of a process of categorizing fields;

FIG. 10 depicts a schematic representation of a process used to manage a patient's pharmaceuticals.

### DETAILED DESCRIPTION OF THE INVENTION

Referring now to the drawings which depict different embodiments consistent with the present invention, wherever possible, the same reference numbers will be used throughout the drawings and the following description to refer to the same or like parts.

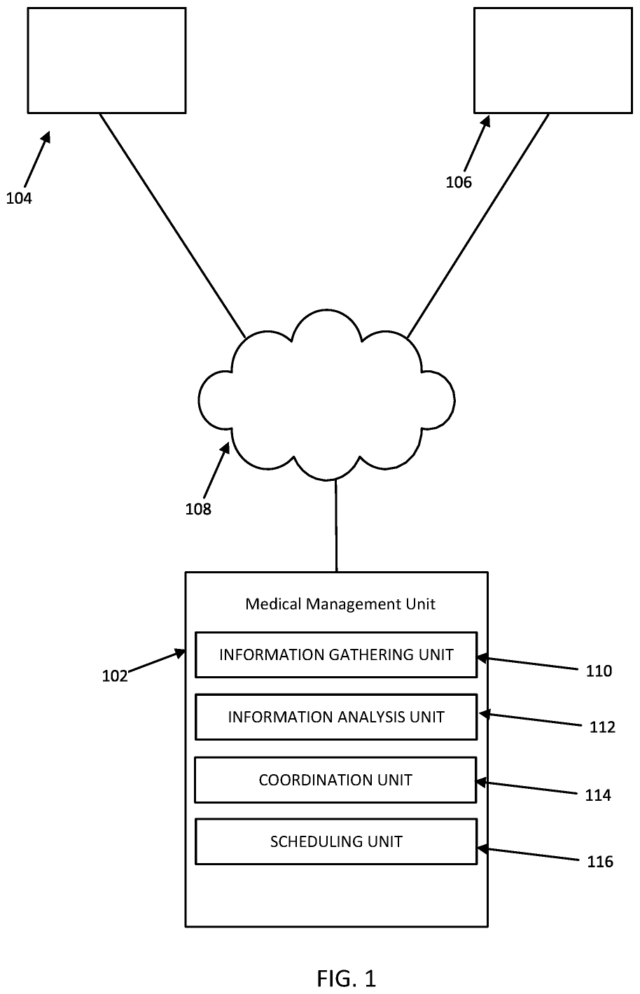

FIG. 1 depicts one embodiment of an integrated medical management system 100 consistent with the present invention. The integrated medical management system 100 includes a medical management unit 102, a communication device 1 104, a communication device 2 106 each communicatively connected via a network 108. The medical management unit 102 further includes an information gathering unit 110, an information analysis unit 112, a coordination unit 114 and a scheduling unit 116.

The information gathering unit 110 and information analysis unit 112 may be embodied by one or more servers. Alternatively, each of the coordination unit 114 and scheduling unit 116 may be implemented using any combination of hardware and software, whether as incorporated in a single device or as a functionally distributed across multiple platforms and devices.

In one embodiment, the network 108 is a cellular network, a TCP/IP network, or any other suitable network topology. In another embodiment, the row identification device may be servers, workstations, network appliances or any other suitable data storage devices. In another embodiment, the communication devices 104 and 106 may be any combination of cellular phones, telephones, personal data assistants, or any other suitable communication devices. In one embodiment, the network 102 may be any private or public communication network known to one skilled in the art such as a local area network (“LAN”), wide area network (“WAN”), peer-to-peer network, cellular network or any suitable network, using standard communication protocols. The network 108 may include hardwired as well as wireless branches. The information gathering unit 112 may include a digital camera.

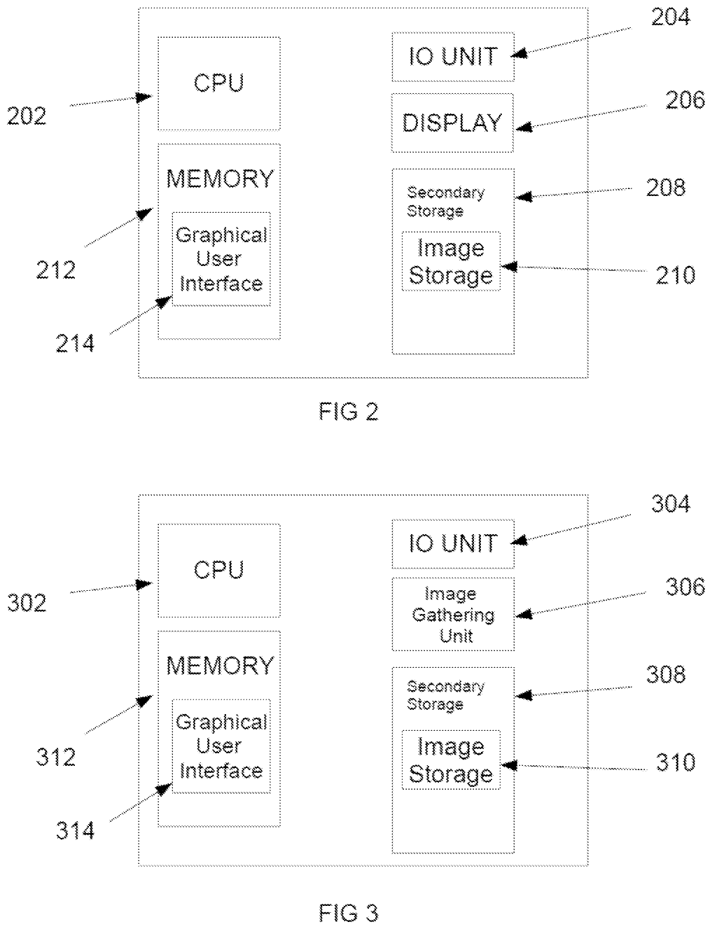

FIG. 2 depicts one embodiment of a medical management unit 102. The agricultural analysis unit 102 includes a network I/O device 204, a processor 202, a display 206 and a secondary storage 208 running image storage unit 210 and a memory 212 running a graphical user interface 214. The information analysis unit 112, operating in memory 208 of the medical management unit 102, is operatively configured to receive an image from the network I/O device 204. In one embodiment, the processor 202 may be a central processing unit (“CPU”), an application specific integrated circuit (“ASIC”), a microprocessor or any other suitable processing device. The memory 212 may include a hard disk, random access memory, cache, removable media drive, mass storage or configuration suitable as storage for data, instructions, and information. In one embodiment, the memory 208 and processor 202 may be integrated. The memory may use any type of volatile or non-volatile storage techniques and mediums. The network I/O line 204 device may be a network interface card, a cellular interface card, a plain old telephone service (“POTS”) interface card, an ASCII interface card, or any other suitable network interface device. The coordination unit 114 may be a compiled program running on a server, a process running on a microprocessor or any other suitable port control software.

FIG. 3 depicts one embodiment of a communication device 104/106 consistent with the present invention. The communication device 104/1106 includes a processor 302, a network I/O Unit 304, an image capture unit 306, a secondary storage unit 308 including an image storage device 310, and memory 312 running a graphical user interface 314. In one embodiment, the processor 302 may be a central processing unit (“CPU”), an application specific integrated circuit (“ASIC”), a microprocessor or any other suitable processing device. The memory 312 may include a hard disk, random access memory, cache, removable media drive, mass storage or configuration suitable as storage for data, instructions, and information. In one embodiment, the memory 312 and processor 302 may be integrated. The memory may use any type of volatile or non-volatile storage techniques and mediums. The network I/O device 304 may be a network interface card, a plain old telephone service (“POTS”) interface card, an ASCII interface card, or any other suitable network interface device.

In one embodiment, the network 108 may be any private or public communication network known to one skilled in the art such as a Local Area Network (“LAN”), Wide Area Network (“WAN”), Peer-to-Peer Network, Cellular network or any suitable network, using standard communication protocols. The network 108 may include hardwired as well as wireless branches.

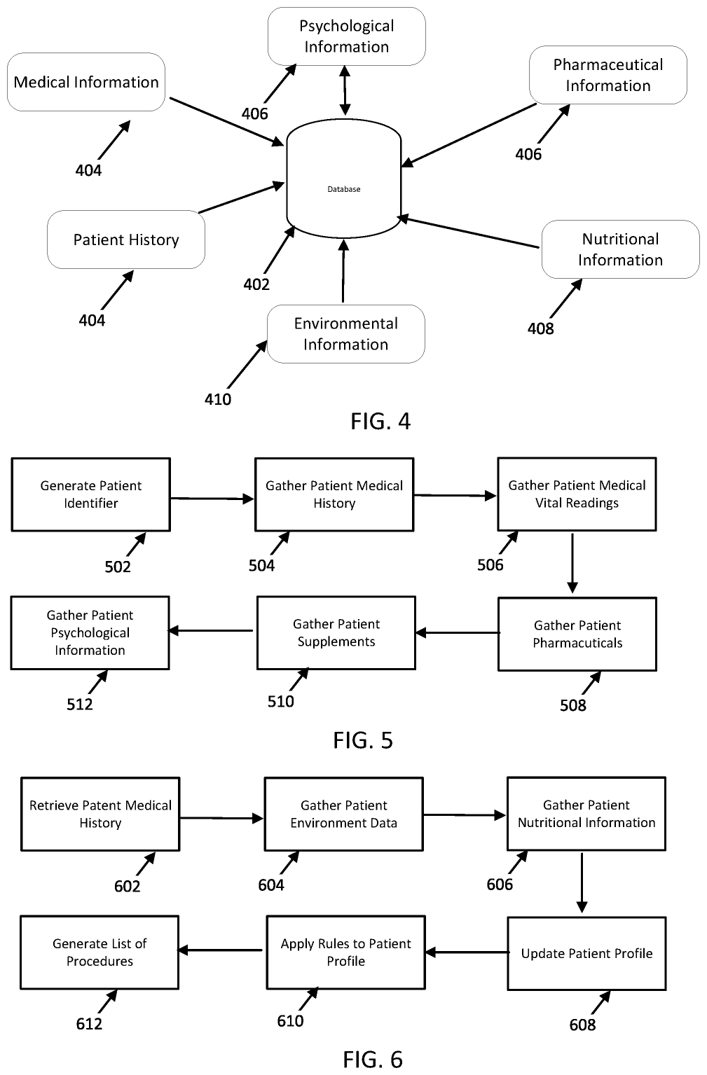

FIG. 4 depicts a schematic representation of the components of the integrated medical management system 100. The integrated medical management system 100 includes a central database 402 that collects information from a plurality of sources. Medical information 404 is gathered from a patient's medical records and is updated regularly at each medical visits. Psychological information 404 is gathered from patient records and from observations from a trained psychological professional. Pharmaceutical information 406 is gathered from the patient directly and from patient records. Nutritional information 408 is gathered from patent surveys, observations and blood tests. Environmental information 410 is gathered from observations and patient questionnaires. Environmental information 410 includes, but is not limited to, climate information, living and work conditions, water and soil conditions in the area and geographical information. Patient history 404 is gathered from the patient and includes, but is not limited to, a patients medical history and family medical history.

FIG. 5 depicts a schematic representation of an intake process for a patient. In step 502, a unique patient identifier is generated for the patient. In step 504, a patient's medical history is gathered from questionnaires, other physician's offices and hospital records. In step 506, a patient's vital readings are gathered. The vital readings include, btu are not limited to, blood pressure, heart rate, temperature, blood components, micro-nutrients and any other physical attribute of the patient. In step 508, a listing of the patient's pharmaceuticals is gathered from the patient and from the patient's pharmacy. In step 510, a listing of patient supplements is gathered from the patient. The supplements include, but are not limited to, vitamins and minerals. In one embodiment, the supplements include the brand name used and the dosage. In step 512, psychological information is gathered by a trained psychological professional.

FIG. 6 depicts a schematic representation of a process used to enhance a patent profile. In step 602, a patient's medical history is retrieved from the database. In step 604, environmental information related to the patient is gathered. Environmental information includes any information related to the environment where the patient lives and works. Environmental information may be gathered directly from the patient, by visiting the patient's residence, using statistical information on a region, using demographic information on a region or any other method of gathering environmental information. In step 606, information related to the patient's nutritional habits are gathered. In one embodiment, a micro-nutrient blood test is administered to the patient and the results of the blood test are stored in the database. In step 608, the patient profile is updated with the new information. In step 610, the information in the patient profile is reviewed using pre-determined rules. The rules are guidelines to recommend specific tests and procedures based on the information in the patient profile. In one embodiment, the rules dynamically change based on previously applied rules. In step 612, a list of procedures and tests is generated based on the application of the rules.

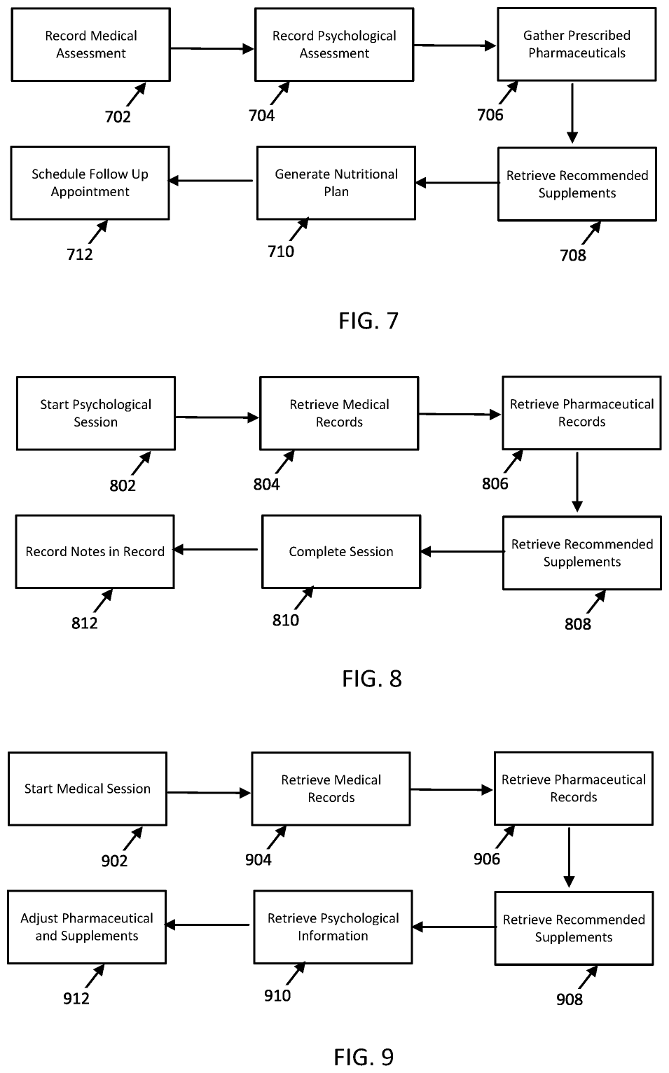

FIG. 7 depicts a schematic representation of a process used generate a health plan. In step 702, a medical assessment is performed on the patient. The medical assessment includes, but is not limited to, a physical examination, blood tests, micro-nutrient tests, physical ability test and any other wellness test In step 706, the list of the patient's pharmaceuticals is retrieved. In step 708, a listing of the patients supplements is gathered including the brand and dosage of each supplement. In step 710, a general nutrition plan is generated based on the medical assessment. In step 712, a follow up visit is scheduled for the patient.

FIG. 8 depicts a schematic representation of a process used to gather psychological information on a patient. In step 802, a psychological assessment is initiated between a psychological professional and a patient. In step 804, the patient's medical record is retrieved from the databases. The medical record includes all information on the patient including, but not limited to, environmental information, nutritional information, and medical assessments. In step 806, the pharmaceutical information on the patient is retrieved. In step 808, the supplement information is retrieved. In step 810, the professional reviews the information on the patient and assesses the patient's current psychological state. In one embodiment, the professional compares the patient's past psychological states with the current state and records any variations in the patient's record. In step 812, the professional records detailed notes on the session in the patient record. The professional may also update environmental records, nutritional records and any other records related to the current visible state of the patient.

FIG. 9 depicts a schematic representation of a process of categorizing fields. In step 902, a medical session with a physician is started. In step 904, the patient's medical record is retrieved from the databases. The medical record includes all information on the patient including, but not limited to, environmental information, nutritional information, and medical assessments. In step 906, the patient pharmaceutical records are retrieved. In step 908, the patient supplement records are retrieved. In step 910, the patient psychological records are retrieved. In step 912, the physician reviews all the retrieved information and performs a medical assessment of the patient. Using the information from gathered, the physician adjusts medication, the patient nutritional plan, supplements and any other aspect of the patient's plan. The physician also recommends additional treatments based on the assessment by the psychologist and pharmacist.

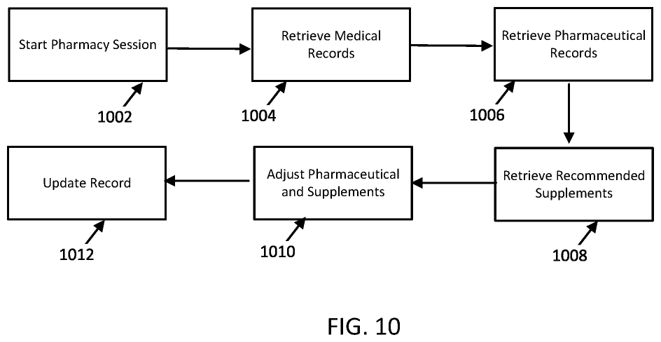

FIG. 10 depicts a schematic representation of a process used to manage a patient's pharmaceuticals. In step 1002, a session is started with the patient and a pharmacist. In step 1004, the patient's medical record is retrieved from the databases. The medical record includes all information on the patient including, but not limited to, environmental information, nutritional information, and medical assessments. In step 1006, the patient's pharmaceutical records are retrieved. In step 1008, the patient's supplements are retrieved. In step 1008, the pharmacist reviews the information from the psychologist and physician to determine the best pharmaceutical course for the patient. The pharmacist may consult with the physician and psychologist to adjust medication levels and types and to adjust supplement types and levels based on the patient's physical and mental condition. In step 1012, the patient's record is updated to reflect any changes and observations.

Using an integrated approach enhances patient treatments and outcomes. In addition, various artificial intelligence models can be applied to groups of similar patient information to generate correlations between different patients. In one embodiment, the system tracks groups of patients taking one or more supplements and records changes in patient's mental and physical condition. In another embodiment, the system may generate a virtual patient that incorporates various characteristics of groups of patients. The system can apply different treatments and nutritional plans to the virtual patient to determine the best potential treatments for different ailments.

While various embodiments of the present invention have been described, it will be apparent to those of skill in the art that many more embodiments and implementations are possible that are within the scope of this invention. Accordingly, the present invention is not to be restricted except in light of the attached claims and their equivalents.

What is claimed:

1. A medical management system including:
an information gathering unit that gathers information on a patient;
an information analysis unit that analyzes the information of each patient;
a coordination unit that disseminates the patient information to different professionals;
a scheduling unit that coordinates the patient visit schedules,
wherein,
the patient information includes medical, psychological and pharmaceutical assessments.

2. The medical management system of claim 1 wherein environmental information related to the patient is gathered.

3. The medical management system of claim 2, wherein the environmental information includes information on the environment where the patient works and lives.

4. The medical management system of claim 2, wherein the environmental information includes demographic information related to the area where the patient works and lives.

5. The medical management system of claim 1, wherein the patient information includes a listing of all pharmaceuticals and supplements taken by the patient.

6. The medical management system of claim 1, wherein the patient information includes psychological assessments made by a trained psychologist.

7. The medical management system of claim 1, wherein the patient information is regularly updated by medical professionals.

8. The medical management system of claim 1, wherein the patient information includes information on the nutritional status of the patient.

9. The medical management system of claim 1, wherein the patient information is normalized to similar patients.

10. The medical management system of claim 1, wherein a pharmacist reviews the patient medication and makes adjustments based on information from a psychologist and physician.

11. The method of managing a patient's overall health including the steps of:
gathering information on a patent an information gathering unit;
analyzing the patient information via an information analysis unit;
disseminating patient information to different professionals via a coordination unit;
coordinating patient visits via a scheduling unit,
wherein,
the patient information includes medical, psychological and pharmaceutical assessments.

12. The method of claim 11 wherein environmental information related to the patient is gathered.

13. The method of claim 12, wherein the environmental information includes information on the environment where the patient works and lives.

14. The method of claim 12, wherein the environmental information includes demographic information related to the area where the patient works and lives.

15. The method of claim 11, wherein the patient information includes a listing of all pharmaceuticals and supplements taken by the patient.

16. The method of claim 11, wherein the patient information includes psychological assessments made by a trained psychologist.

17. The method of claim 11, wherein the patient information is regularly updated by medical professionals.

18. The method of claim 11, wherein the patient information includes information on the nutritional status of the patient.

19. The method of claim 11, wherein the patient information is normalized to similar patients.

20. The method of claim 11, wherein a pharmacist reviews the patient medication and makes adjustments based on information from a psychologist and physician.
